(19) United States
(12) Patent Application Publication
(10) Pub. No.: US 2002/0077534 A1
(43) Pub. Date: Jun. 20, 2002

(54) Method and system for initiating activity based on sensed electrophysiological data

(75) Inventor: Donald R. DuRousseau, Purcellville, VA (US)

Correspondence Address: PEPPER HAMILTON LLP, Attn: James M. Singer, 50th Floor, 500 Grant Street, Pittsburgh, PA, 15219, US

(73) Assignee: Human Bionics LLC

(21) Appl. No.: 10/028,902

(22) Filed: Dec. 18, 2001

Related U.S. Application Data
(60) Provisional application No. 60/255,904, filed on Dec. 18, 2000.

(51) Int. Cl. A61B 5/00

(52) U.S. Cl. 600/300; 600/544; 600/546; 600/595; 340/825.19; 128/925

(57) ABSTRACT

A hands-free human-machine interface uses body position, limb motion, speech signals, and/or changes in the operator's level of cognition and/or stress to control the user interface of an interactive system. Signals are acquired from mental and/or physical processes, such as brainwaves, eye, heart, and muscle activities, larynx activity, body position and motion changes, and stress indicating measures. The signals are measured and processed to replace a hand-operated mouse, keypad, joystick, video game, or other controls with a motion-based gestural interface that works, optionally in conjunction with a larynx activated speech processor. For disabled individuals without sufficient dexterity and speech capacity, multimodal neuroanalysis will reveal intended movements and these will be used to operate an imagined mouse or keypad.

### PRIORITY

This application claims priority to the provisional U.S. patent application entitled Computer Interface, filed Dec. 18, 2000, having a Ser. No. 60/255,904, the disclosure of which is hereby incorporated by reference.

### FIELD OF TEE INVENTION

The present invention relates generally to biofeedback devices and systems. More particularly, the present invention relates to a mobile method and system for processing signals from the human brain and/or body. The processed signals can be used to operate various computer based applications by replacing or enhancing the control signals from speech recognition systems and other hand-operated input devices like the keypad, mouse, joystick, or video game controller.

### BACKGROUND OF THE INVENTION

Conventional interactive applications rely on control input from devices, such as the keyboard, mouse, joystick, game controller or continuous speech processor. These devices are used globally to communicate our intentions about how we want to interact with a computer's operating system, typically via the graphical user interface (GUI) of the host application, which in turn communicates with the application program interface (API) to produce the intended result. In all cases, electronic signal processing is employed to detect the user's intentions (e.g., a click of a mouse button, a push of a keypad, or use of appropriate words such as “Open—New File”) and then to influence, augment, or otherwise control the operation of an interactive program and/or device.

Conventional human-computer interfaces are limited, however, in that they require a human to physically interact with the device, such as by using a finger to press a button. Thus, persons with disabilities, as well as persons working in conditions where hands are required for other tasks, do not have an adequate interface with which to control their computer systems.

Prior attempts to solve this problem have included speech processing systems for voice activation. However, voice activation is often not desirable because of many use-related limitations, including but not limited to poor operation in noisy environments, inappropriateness in public places, and difficulty of use by those with speech and hearing problems. Some researchers have attempted to use head and eye movement schemes to move a cursor around on a CRT screen. Such methods are limited in control functionality and require additional measures to provide a robust control interface. Others in the brain-computer interface community have investigated the use of imagined movements as a type of control signal. Other groups are implanting electrodes directly into the motor cortex of apes, in an attempt to illicit control signals directly from the brain. Such methods are clearly impractical for general use by humans. In addition, the methods are asynchronous and lack sufficient multimodal indicators, other than the electroencephalogram (EEG) signals, to ensure the accuracy of the intended control outputs.

Additional prior attempts to provide human-computer interfaces include works such as those described in U.S. Pat. No. 4,461,301 to Ochs, U.S. Pat. No. 4,926,969 to Wright et al., and U.S. Pat. No. 5,447,166 to Gevins. However, the prior attempts measure only the EEG and do not rely on a combination of physiological signals from the brain and body to affect the control of interactive systems. In particular, the prior attempts do not rely on multimodal signal processing methods to measure the user's real or imagined control intentions. Nor do they work within the intended host system as an embedded processor that directly interacts with the host's operating system.

Thus, the limitations of controlling interactive systems with hand-operated and/or loudly-spoken-language controls are obvious, while the potential benefits of novel volitional computer interfaces are limited only by the imagination. Reliable hands-free mind- and body-driven control over interactive hardware and software systems would offer everyone, including those suffering from disabling conditions and those working in areas requiring constant use of their hands, drastically improved access to communication, education, entertainment, and mobility systems.

Accordingly, it is desirable to provide an improved human-computer interface (HCI) having many of the same capabilities as conventional input devices, except the novel interface does not require hand-operated electromechanical controls or microphone-based speech processors.

### SUMMARY OF THE INVENTION

It is therefore a feature and advantage of the present invention to provide an improved human-computer interface, referred to herein as a Bio-adaptive User Interface (BUI™) system, having many of the same capabilities as a conventional input device, but which is hands-free and does not require hand operated electromechanical controls or microphone-based speech processing methods.

The above and other features and advantages are achieved using a novel BUI as herein disclosed. In accordance with one embodiment of the present invention, a method of analyzing a signal indicative of detecting an intended event from human sensing data includes the steps of: (i) receiving a signal indicative of physical or mental activity of a human; (ii) using adaptive neural network based pattern recognition to identify and quantify a change in the signal; (iii) classifying the signal according to a response index to yield a classified signal; (iv) comparing the classified signal to data contained in a response database to identify a response that corresponds to the classified signal; and (v) delivering an instruction to implement the response.

Optionally, the method includes processing the signal to identify one or more of a cognitive state of the human, a stress level of the human, physical movement of the human body, body position changes of the human, and motion of the larynx of the human. Also optionally, the using step may include identifying at least one factor corresponding to the signal and weighting the signal in accordance to the at least one factor, the receiving step may include receiving a signal from one or more sensors that are in direct or indirect contact with the human, and the classifying step may include classifying the signal according to one of an electrophysiological index, a position index, or a movement index. Further, wherein the delivering step may include delivering a computer program instruction to a computing device via a computer interface

Also optionally, the comparing step may be performed using at least one fast fuzzy clarifier.

In addition, the method may be implemented by computer program instructions stored on a carrier such as a computer memory or other type of integrated circuit.

There have thus been outlined the more important features of the invention in order that the detailed description thereof that follows may be better understood, and in order that the present contribution to the art may be better appreciated. There are, of course, additional features of the invention that will be described below and which will form the subject matter of the claims appended hereto.

In this respect, before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Hence, it is to be understood that the phraseology and terminology employed herein, as well as in the abstract, are for the purpose of description and should not be regarded as limiting.

As such, those skilled in the art will appreciate that the conception upon which this disclosure is based may readily be utilized as a basis for the designing of other structures, methods, and systems for carrying out the several purposes of the present invention. It is important, therefore, that the claims be regarded as including such equivalent constructions insofar as they do not depart from the spirit and scope of the present invention.

### BRIEF DESCRIPTION OF THE DRAWINGS

FIG. 1 illustrates several hardware elements of a preferred system embodiment of the invention.

FIG. 2 is a block diagram illustrating the signal processing path implemented by the BUI Library method of the present invention.

FIG. 3 provides a perspective view illustrating several elements of a class-dependent heuristic data architecture used in a preferred embodiment of the present invention.

FIG. 4 is a block diagram illustrating exemplary elements of a digital processor, memory, and other electronic hardware.

### DETAILED DESCRIPTION OF PREFERRED EMBODIMENTS OF THE INVENTION

A preferred embodiment of the present invention provides an improved human-computer interface (HCI) having many of the same capabilities as a conventional input device, like a keyboard, mouse or speech processor, but which does not require hand operated mechanical controls or traditional microphone-based voice processors. A preferred embodiment may rely on physiological signals from the brain and body, as well as from motion and vibration signals from the larynx, to control interactive systems and devices. The invention works within a host environment (i.e., a desktop or body-worn PC running an interactive target application) and preferably replaces the electromechanical input device used to manipulate the program's graphical user interface (GUI). The preferred embodiment of the present invention provides a psychometric HCI that can be packaged as a software developers kit (SDK) to allow universal use of the method. To install the interface, a driver preferably will be used to load a BodyMouse™ controller that uses cognitive and stress related signals from the brain and body and/or motion information from the larynx in place of awkward hand manipulations and/or loudly spoken language.

The present invention relates to a mobile method and system for processing signals from the human brain and/or body using some or all of the following features: (i) a positioning system that locates sensors and transducers on or near the body; (ii) a medical grade ambulatory physiological recorder; and (iii) a computing device that can wirelessly transmit physiological and video image data onto a World Wide Web site

A purpose of the present invention is to use changes in psychometric information received via body-mounted sensors and/or transducers to detect and measure volitional mental and physical activity and derive control signals sufficient to communicate the user's intentions to an interactive host application. The invention thus provides a human-machine communication system for facilitating hands-free control over interactive applications used in communication, entertainment, education, and medicine.

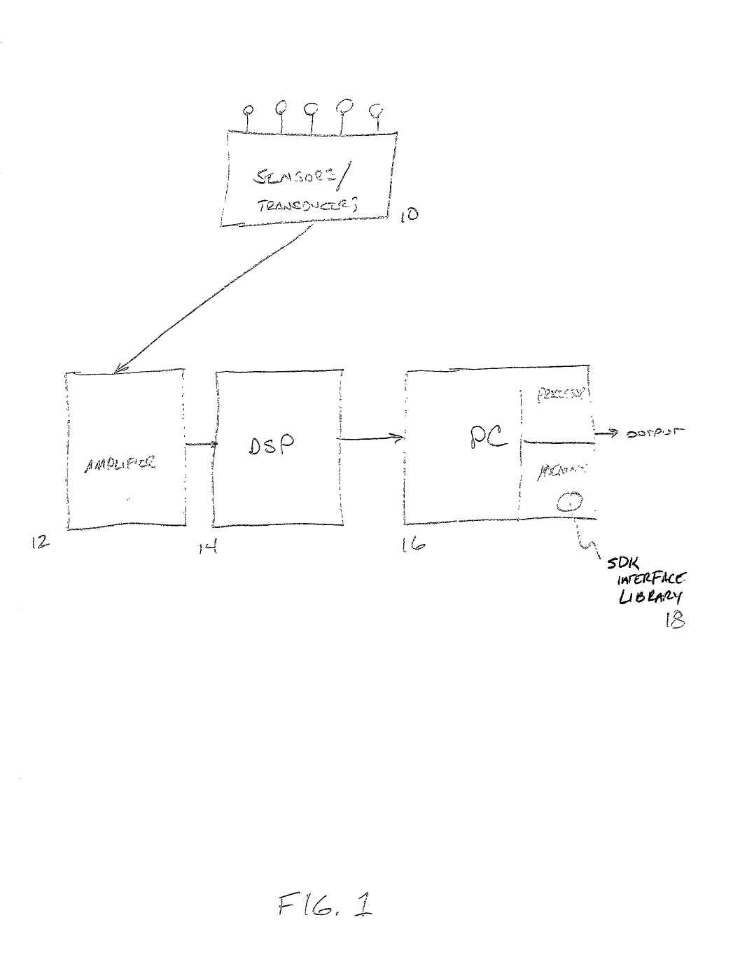

A preferred system embodiment of the present invention is illustrated in FIG. 1. As illustrated in FIG. 1, the system includes at least three primary parts: (1) a wearable sensor placement unit 10 (preferably stealthy and easy to don), which also locates several transducer devices, such as that disclosed in U.S. Pat. No. 5,038,782, to Gevins et al, which is incorporated herein by reference; (2) an integrated multichannel amplifier 12, a digital signal processing (DSP) unit 14 and a personal computer (PC) 16, all small enough to wear on the human body; and (3) a self-contained BUI™ Library 18 of software subroutines, which comprise the signal-processing methods that measure and quantify numerous psychometric indices derived from the operator's mental, physical, and movement related activities to provide volitional control of the GUI interface. Preferably, the BUI Library 18 component of the present invention can be embodied in such a way as to provide a stand-alone SDK that gives application makers a universal programming interface to embed cognitive, enhanced speech, and gesture control capabilities within all types of interactive software and hardware applications. The PC 16 contains both a processing device and a memory. Thus, optionally, a subset of the BUI Library 18 can be provided within the interactive application running on the PC 16 as an embedded controller to process signals and provide interoperability with the program's application program interface (API). The amplifier 12 and/or the DSP 14 may also be included within the housing of the PC 16 to miniaturize the overall system size, thereby producing an integrated digital acquisition unit 17. In the preferred embodiment, the host application (to be controlled by signals received from the sensor placement unit 10) is installed on the PC 16, although in alternate embodiments the controlled application may be operating on an external computing device that communicates with the PC 16 through any communication method such as direct wiring, telephone connections, wireless connections, and/or the Internet or other computer networks.

Preferably, the sensor placement unit 10 is capable of receiving electrophysiology in various forms, such as EEG signals, electromyographic (EMG) signals, electrooculographic (EOG) signals, electrocardiographic (ECG) signals, as well as body position, motion and acceleration, vibration, skin conductance, respiration, temperature, and other physical measurements from transducers. The system must be capable of delivering uncontaminated or substantially uncomtaminated signals to the digital acquisition unit 17 to derive meaningful control signals to manipulate the API, thus providing some or all of the functions of conventional natural language and/or electromechanical controllers.

The sensor placement unit 10 preferably exhibits some or all of the followings features: (1) it has relatively few input types (preferably less than eighteen, but it may include as many as forty or more) and can be quickly located on the body of the operator; (2) it positions biophysical (EEG, ECG, EMG, etc.) surface electrodes, and transducers for acquiring vibration, galvanic skin response (GSR), respiration, oximetry, motion, position, acceleration, load, and/or resistance, etc; (3) the sensor attachments are unobtrusive and easy (for example, easy enough for a child of age ten) to apply (preferably in less than three minutes); (4) the sensor placement unit 10 accommodates multiple combinations of electrodes and/or transducers; (5) the surface electrodes use reusable and/or replaceable tacky-gel electrolyte plugs for ease and cleanliness; and (6) EEG, EOG, ECG, and EMG electrodes may be positioned simultaneously and instantly on a human head by a single positioning device.

In a preferred embodiment, the sensor placement unit 10 comprises a stealthy EEG placement system capable of also locating EOG, EMG, ECG, vibration, GSR, respiration, acceleration, motion and/or other sensors on the head and body. The sensor and transducer positioning straps should attach quickly and carry more than one type of sensor or transducer. In a preferred embodiment, the unit will include four EEG sensors, two EOG sensors, four EMG sensors, and a combination of vibration, acceleration, GSR, and position measures. However, any combination of numbers and types of sensors and transducers may be used, depending on the application.

Each sensor can preferably be applied with the use of a semi-dry electrolyte plug with exceptional impedance lowering capabilities. In a preferred embodiment, a single electrolyte plug will be placed onto each surface electrode and works by enabling instantaneous collection of signals from the skin. The electrolyte plugs will be replaceable, and they may be used to rapidly record from sensors without substantial, and preferably without any, abrasion or preparation of the skin. The electrolyte plugs should be removable to eliminate the need to immediately wash and disinfect the sensor placement unit 10 in liquids. By eliminating the need to wash the system after each use, the sensor placement system 10 will be ideal for use in the home or office.

The sensor placement unit 10 preferably communicates with the digital acquisition unit 17, consisting of an amplifier 12, DSP 14, and PC 16, and the entire assembly exhibits some or all of the following features: (1) it is small enough to wear on the body; (2) it has received Conformite Europeene (CE) marking and/or International Standards Organization (ISO) certification and is approved for use as a medical device in the United States; (3) it processes several, preferably at least sixteen and not more than forty, multipurpose channels, plus dedicated event and video channels; (4) it provides a universal interface that accepts input from various sensors and powers several body-mounted transducers; (5) it is capable of high-speed digital signal processing of the EEG, EOG, ECG, EMG and/or other physiological signals, as well as analyzing measurements from a host of transducer devices; and (6) it offers a full suite of signal processing software for viewing and analyzing the incoming data in real time.

The digital acquisition unit 17, working with the BUI Library 18, preferably exhibits some or all of the following features: (1) it provides an internal DSP system capable of performing real time cognitive, stress, and motion assessment of continuous signals (such ;as EEG, EMG, vibration, acceleration, etc.) and generating spatio-temporal indexes, linear data transforms, and/or normalized data results. Processing requirements may include (i) EOG detection and artifact correction; (ii) spatial, frequency and/or wavelet filtering; (iii) boundary element modeling (BEM) and finite element modeling (FEM) source localization; (iv) adaptive neural network pattern recognition and classification; (v) fast fuzzy cluster feature analysis methods; (vi) real time generation of an output control signal derived from measures that may include (a) analysis of motion data such as vibration, acceleration, force, load, position, angle, incline and/or other such measures; (b) analysis of psychophysiological stress related data such as pupil motion, heart rate, blink rate, skin conductance, temperature, respiration, blood flow, pulse, and/or other such measures; (c) spatial, temporal, frequency, and wavelet filtering of continuous physiological waveforms; (d) BEM and FEM based activity localization and reconstruction; (e) adaptive neural network pattern recognition and classification; and (f) fast fuzzy cluster feature extraction and analysis methods.

The data interface between the sensor placement system 10 and host PC 16 can be accomplished in a number of ways. These include a direct (medically isolated) connection, or connection such as via serial, parallel, SCSI, USB, Ethernet, or Firewire ports. Alternatively, the data transmission from the sensor placement system 10 may be indirect, such as over a wireless Internet connection using an RF or IR link to a network card in the PCMCIA bay of the wearable computer. To meet the hardware/software interface requirements, multiple interconnect options are preferably maintained to offer the greatest flexibility of use under any conditions. The software portion of the interface is preferably operated through an application program interface (API) that lets the user select the mode of operation of the hands-free controller by defining Physical Activity Sets (control templates), and launching the chosen application.

The invention also uses a unique processing method, sometimes referred to herein as a Bio-adaptive User Interface™ method, that includes some or all of the following features:

(1) processing of one or more sets of indices relating changes in mental and physical activity in terms of control output signals used for the purpose of communicating the intentions of the user to operate an interactive application without the use of hand operated mechanical devices, or microphone-based auditory speech processors;

(2) processing of one or more sets of indices relating changes in larynx vibratory patterns and associated EMG activity patterns from the controlling muscles in terms of control output signals used for the purpose of communicating the intentions of the user to operate an interactive application without the use of hand operated mechanical devices, or microphone-based auditory speech processors;

(3) the processing of psychophysiological and larynx activation signals using linear and non-linear analytical methods, including automated neural network and fast fuzzy cluster based pattern recognition, classification, and feature extraction methods that fit indices (based on changes within each signal measured) to sets of Activity Templates that provide predetermined control output signals, (e.g. ,a signal that looks like the press of a keypad or the click of the “Left” mouse button);

(4) identification of specific sets of indices within Activity Templates, using adaptive neural network (ANN) and fast fuzzy cluster methods to derive weighting functions applied to determine the greatest contribution associated with a particular class of Library Functions. A programming environment allowing developers to use BUI™ Library capabilities will provide access to signal-processing subroutines via an SDK programming architecture;

(5) Class Libraries that are defined with application rules governing the hardware and software interoperability requirements for a particular class of interactive application, such as a mobile phone, entertainment unit, distributed learning console and/or medical device;

(6) an embeddable SDK kernel that delivers a library of real time function calls, each associated with a particular set of Activity Templates, where combinations of template outputs look to the host software like the control signals from any voice processor, keyboard, mouse, joystick or game controller;

(7) adaptive weighting (and/or other similar methods) to selectively choose a preferred set of Activity Templates, based on an adjustable threshold, to provide the most reliable control scheme for a particular class of interactive application;

(8) having a receiving library that accepts feedback from host applications via Response Templates, which update the selection criteria used to qualify the best fitting set of Physical Activity Set; and

(9) a means to adaptively re-weight the Physical Activity Set contributions to (a particular control signal output, based on updated Response Template information from the host application (allows adjustment and refinement of the control signal outputs, like a form of calibration).

Preferably, the overall system architecture is built upon a heuristic rule set, which governs the usage of the Activity and Response Templates and works like an embeddable operating system (OS) within the host program; handling the massaging between the application's API and the BUI™ Library subroutines. To train the embeddable OS within the host application, the “OS kernel” is preferably tied to a menu-driven query protocol to establish user specific criteria and train the ANN pattern recognition network used for delivering feedback information.

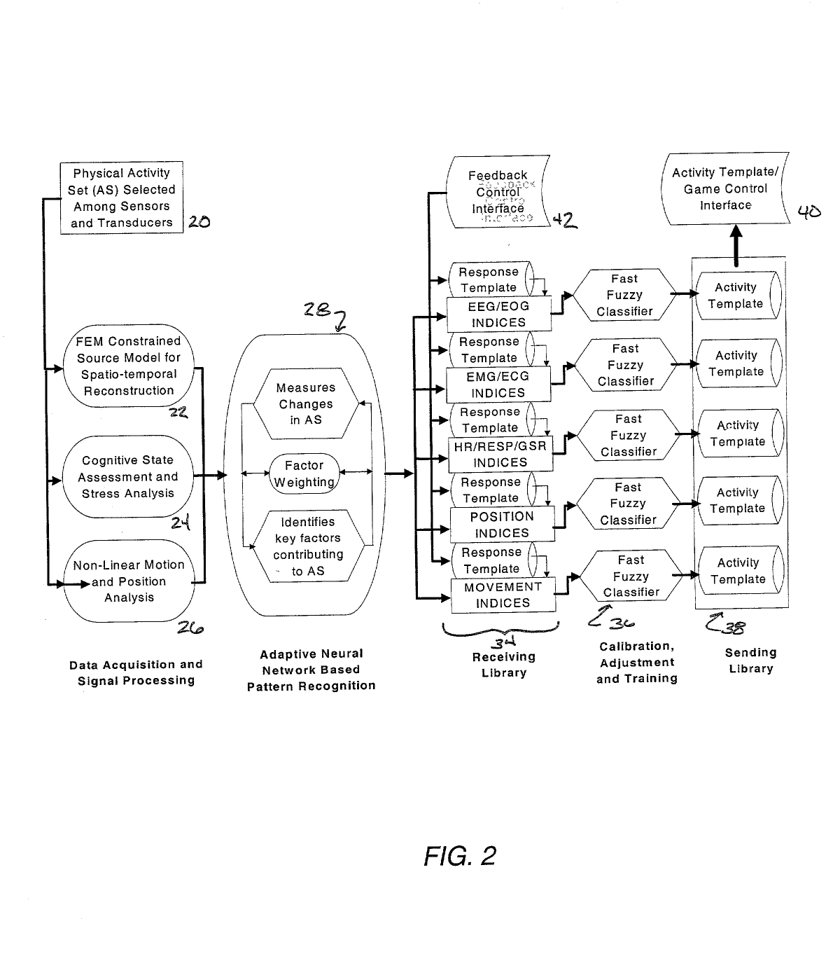

FIG. 2 is a block diagram representation of the present BUI™ Library invention. The invention combines cognitive, stress, and/or larynx processing with limb and body motion analysis, to deliver a hands-free computing system interface. The BUI™ method applies user selected Physical Activity Sets (step 20), which include details of the sensors and transducers needed to collect the appropriate brain and body activities required for a particular application. For example, in a game control application sensors would collect brain, muscle, and heart signals, while transducers detect motion of the limbs, fingers, and other body parts. Then, through novel use of regionally constrained spatio-temporal mapping and source localization methods (step 22), cognitive-state and stress assessment techniques (step 24), and non-linear motion-position analyses (step 26), these signal processing results are fed into an ANN classifier and feature extractor (step 28).

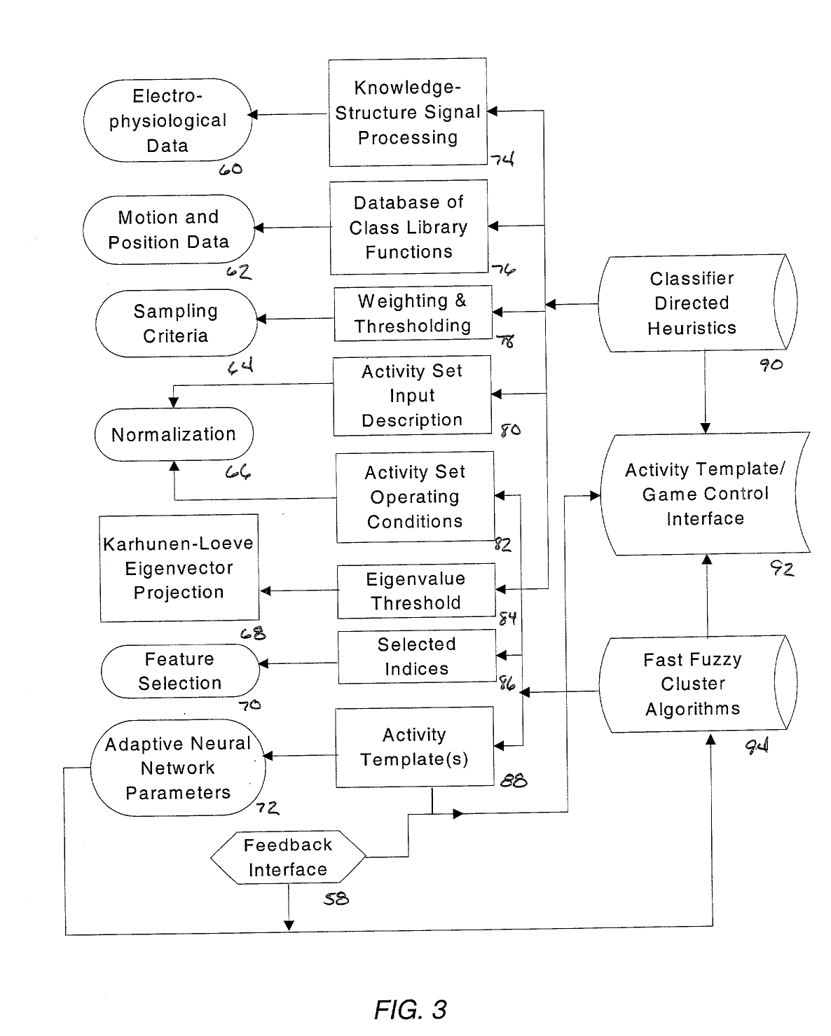

ANN based algorithms (step 28) apply classifier-directed pattern recognition techniques to identify and measure specific changes in each input signal and derive an index of the relative strength of that change. A rule-based hierarchical database structure, or “Class Library Description” (detailed in FIG. 3) describes the relevant features within each signal and a weighting function to go with each feature. A self-learning heuristic algorithm, used as a “Receiving Library” (step 34) governs the use and reweighting criteria for each feature, maintains the database of feature indexes, and regulates feedback from the Feedback Control Interface (step 42). The output vectors from the Receiving Library (step 34) are sent through cascades of Fast Fuzzy Classifiers (step 36), which select the most appropriate combination of features necessary to generate a control signal to match an application dependent “Activity Template” (step 40). The value of the Activity Template (i.e., the port value sent to the host API) can be modified by feedback from the host application through the Feedback Control Interface (step 42) and Receiving Library (step 34) by adaptive weighting and thresholding procedures. Calibration, training, and feedback adjustment are performed at the classifier stage (step 36) prior to characterization of the control signal in the “Sending Library” (step 38) and delivery to the embedded OS kernel in the host application via the Activity Template (step 40), which matches the control interface requirements of the API.

Alternatively, the user selected Physical Activity Set (step 20) may include brainwave source signals, cognitive and stress related signals from the brain and body, larynx motion and vibration signals, body motion and position signals, and other signals from sensors and transducers attached to a human. The Physical Activity Set (step 20) indicates the appropriate signal or activity that the user wants to use in controlling the GUI of the interactive application. For example, the user may select the snap of the fingers on the right hand to mean “press the right button on the mouse”.

Based on the signal features analyzed in steps 22, 24, and/or 26, the BUI system applies ANN-based pattern classification and recognition routines (step 28) to identify changes in the signals specified in the Physical Activity Set. The features of interest may include, for example, shifts in measured activation, frequency, motion, or other index of a signal change. Thus, a change in frequency may be indicative of a body movement, spoken sound, EEG coherence pattern, or other detail of the user's physical condition. Based on the measured changes and the other factors, the factors and changes may be weighted before being sent to a Receiving Library for classification. For example, where the invention is used as a controller for a video game, wherein the user controls a simulated skateboarder, the pattern recognition methods may consider a change in the user's physical movement to have a greater weight than a change in the user's spatio-temporal EEG pattern. In other words, actual limb and body movements of the user may be interpreted to dictate program control, while measures of the user's level of focused attention may be used supplementally to augment game play, say, by making the course tougher, or granting the user's avatar increased or decreased abilities. However, in the case of a quadriplegic, a different Class Library could be used to ignore all but a few signal types and dictate the digital acquisition and processing steps required to detect specific brain activity related to imagined movements of a graphical control system that displays on the screen of the interactive application being run by the user. In this case, only cognitive and stress related signals would be measured.

The signal indices processed and weighted through ANN feature recognition are classified into a data buffer or Receiving Library (step 34), preferably comprising bins of indexes that associate mental and physical activities to sets of output control signals. The Receiving Library separates the appropriate weighted signals so that they may be processed and delivered to the device specific Activity Template (step 40) in order to output the appropriate control signal to operate the host program's API. The signal vectors entered into the Receiving Library are compared to Activity Templates using one or more fast fuzzy clarifiers (step 36) or other appropriate algorithms. The fast fuzzy clarifiers compare the weighted signal data to one or more databases maintained in the Receiving Library (step 34) to identify an appropriate response corresponding to each weighted signal. The processed indicators are then delivered to a Sending Library (step 38) where the contribution of each indicator, as a relevant control output, is measured and classified into an Activity Template that passes control signals, via an embedded OS kernel, to mimic the actions of the mouse, joystick, speech processor, hand held controller, or other control device.

The BUI method also provides for adaptive feedback from the host application through the Response Template that can update signals in the Receiving Library, thus modifying the output vectors to the Sending Library and ultimately to the host application.

A block diagram detailing the operating rules and data interrelationship within the BUI™ Library is shown in FIG. 3. The boxes on the left side of FIG. 3 (boxes 60 through 72) relate to rules that are part of the Physical Activity Set selection process specified in FIG. 2 (step 20). The actions listed in step 52 detail the data relationships and signal processing requirements needed to derive class specific features from the selected signal types, dependent on the type of application (i.e., communication system, training console, game platform, or medical device). The boxes down the center of FIG. 3 (boxes 74 through 88) relate to the data relationships, index-weighting functions, and baseline threshold criteria used in operating the Receiving Library (step 34) and Feedback Control Interface (step 42) of FIG. 2. The boxes on the right side of FIG. 3 (boxes 90 through 94) relate to the data relationships, output control signal characteristics, and device specific interface requirements used in operating the Sending Library (step 38) and Activity Template Control Interface (step 40) of FIG.2. The Feedback Interface box (box 58) provides the data relationships, index-weighting functions, and baseline threshold criteria used in operating the Feedback Control Interface (step 42) of FIG. 2.

The present invention provides several advantages over the prior art. For example, the invention may provide a novel wearable bio-adaptive user interface (BUI™) that utilizes miniaturized ultra-lightweight acquisition and computing electronics and sophisticated signal processing methods to acquire and measure psychometric data under real-world conditions.

A preferred embodiment of the present invention also provides a multichannel sensor placement and signal processing system to record, analyze, and communicate (directly or indirectly) psychophysiological and physical data, as well as stress and movement related information.

A preferred embodiment of the present invention also provides a multichannel sensor placement and signal processing system to record, analyze, and communicate larynx activity, contained in the form of vibration patterns and muscle activation patterns, to provide a silent speech processor that does not use microphone-based auditory signals.

A preferred embodiment of the present invention also provides specially configured sensor and transducer kits packaged to acquire application specific signal sets for communication, entertainment, educational, and medical applications.

A preferred embodiment of the present invention also provides a universal interface to the signal processing system that is modular and allows attachment to many different sensors and transducers.

A preferred embodiment of the present invention also collects, processes and communicates psychometric data over the Internet anywhere in the world to make it available for review or augmentation at a location remote from the operator or patient.

A preferred embodiment of the present invention also provides a BUI™ Library of signal processing methods, which measure and quantify numerous psychometric indices derived from the operator's mental, physical, and movement related efforts to provide hands-free control of the API. For instance, a game application may require the press of the “A Button” on a joystick to cause the character to move left; the BUI™ Library can output the same control signal, except, it is based on a relevant combination of brain and/or body activities rather than movement of buttons on the hand-operated controller.

A preferred embodiment of the present invention also provides a volitional bio-adaptive controller that uses multimodal signal processing methods to replace or supplement the mechanical and/or spoken language input devices that operate the host application's GUI. The BUI™ will provide an alternative method of controlling hardware and software interactions from the existing electromechanical and speech-based input devices and is intended to operate with-in standard operating systems such as, for example, Windows®, UNIX® and LEINX®.

A preferred embodiment of the present invention also provides a volitional bio)adaptive controller that uses multimodal signal processing methods to replace or supplement the mechanical and/or spoken language input devices that operate the graphical user interface (GUI) of the console style game systems. The BUI will provide an alternative method of controlling console-based programs than the existing electromechanical and speech-based input devices and is intended to operate with many conventionally available game consoles, such as Nintendo, N64, Sega Dreamcast, Playstation II and Microsoft's Xbox.

A preferred embodiment of the present invention also provides multimodal signal processing methods that measure and quantify multiple types of psychometric data, and output specific indices that reflect varying levels of the user's mental and physical efforts (e.g., levels of alertness, attention, vigilance, drowsiness, etc.) that can be used to purposely control interactive applications (“volitional control”).

A preferred embodiment of the present invention also provides multimodal signal processing methods that measure and quantify head, limb, body, hand, and/or finger movements, and output specific indices that reflect varying levels of control based on the intentional (or imagined) motion of part, or all of the user's body, intended to purposely control interactive applications (also “volitional control”).

A preferred embodiment of the present invention also provides multimodal signal processing methods that measure and quantify the vibration and muscle activation patterns of the larynx during speech, and more particularly, during whispered speech, and output specific indices that reflect varying levels of control based on the spoken or whispered language content in a manner consistent with existing continuous speech and natural language processing methods.

A preferred embodiment of the present invention also provides a bundling of the BUI™ Library and BodyMouse™ Controller Driver into a software developers kit (SDK) with an embeddable programming environment that allows application makers to use cognitive, gestural, and silent speech controllers to operate their interactive systems.

A preferred embodiment of the present invention also includes, within the SDK, subroutines that allow developers to create software with the ability to instantly modify program operation based on the mental and physical activity of the user.

A preferred embodiment of the present invention also includes, within the software development kit, subroutines that allow developers to create software with the capacity to volitionally control Microprocessor-Based Electromechanical Systems (MEMS) used in restorative and rehabilitation devices.

In a preferred embodiment a single surface electrode, or group of electrodes, may be used to acquire signals from the brain, eyes, skin, heart, muscles, larynx, or, by providing a means to position electrodes and transducers in the appropriate regions on or near the scalp, face, chest, skin, or body. For instance, ubiquitously placed in clothing or included as part of a chair or as a peripheral computing device.

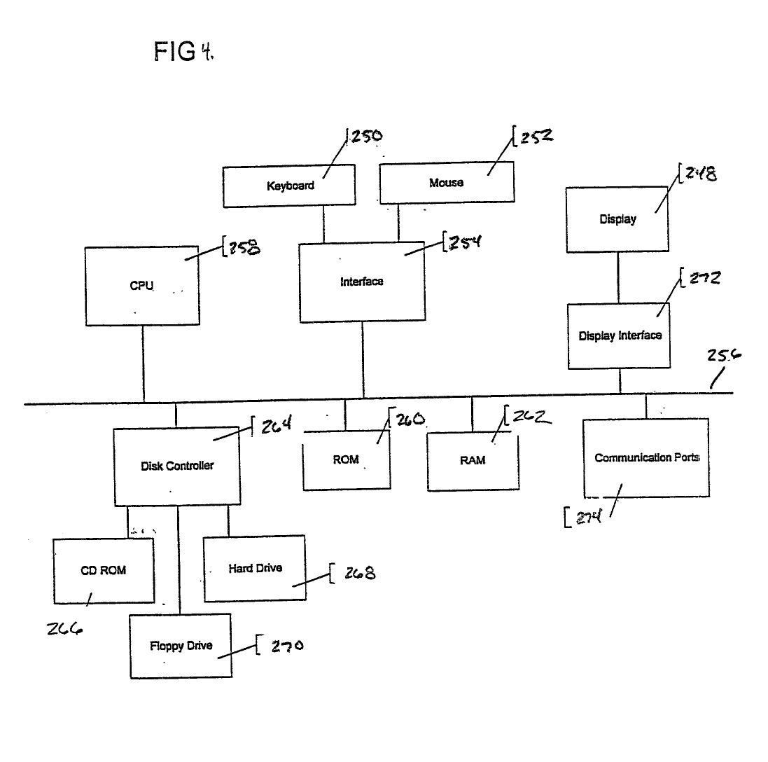

FIG. 4 is a block diagram of exemplary internal hardware that may be used to contain or implement the program instructions of a system embodiment of the present invention. Referring to FIG. 4, a bus 256 serves as the main information highway interconnecting the other illustrated components of the hardware. CPU 258 is the central processing unit of the system, performing calculations and logic operations required to execute a program. Read only memory (ROM) 260 and random access memory (RAM) 262 constitute memory devices.

A disk controller 264 interfaces one or more optional disk drives to the system bus 256. These disk drives may be external or internal floppy disk drives such as 270, external or internal CD-ROM, CD-R, CD-RW or DVD drives such as 266, or external or internal hard drives 268. As indicated previously, these various disk drives and disk controllers are optional devices.

Program instructions may be stored in the ROM 260 and/or the RAM 262. Optionally, program instructions may be stored on a computer readable carrier such as a floppy disk or a digital disk or other recording medium, a communications signal, or a carrier wave.

An optional display interface 272 may permit information from the bus 256 to be displayed on the display 248 in audio, graphic or alphanumeric format. Communication with external devices may optionally occur using various communication ports such as 274.

In addition to the standard computer-type components, the hardware may also include an interface 254 which allows for receipt of data from the sensors or tranducers, and/or other data input devices such as a keyboard 250 or other input device 252 such as a remote control, pointer, mouse, joystick, and/or sensor/transducer input.

The many features and advantages of the invention are apparent from the detailed specification. Thus, the appended claims are intended to cover all such features and advantages of the invention, which fall within the true spirits and scope of the invention. Further, since numerous modifications and variations will readily occur to those skilled in the art, it is not desired to limit the invention to the exact construction and operation illustrated and described. Accordingly, all suitable modifications and equivalents may be included within the scope of the invention.

What is claimed is:

1. A method of analyzing a signal indicative of detecting an intended event from human sensing data, comprising:
receiving a signal indicative of physical or mental activity of a human;
using adaptive neural network based pattern recognition to identify and quantify a change in the signal;
classifying the signal according to a response index to yield a classified signal;
comparing the classified signal to data contained in a response database to identify a response that corresponds to the classified signal; and
delivering an instruction to implement the response.

2. The method of claim 1 further comprising processing the signal to identify one or more of a cognitive state of the human, a stress level of the human, physical movement of the human body, body position changes of the human, and motion of the larynx of the human.

3. The method of claim 1 wherein the using step further comprises:
identifying at least one factor corresponding to the signal; and
weighting the signal in accordance to the at least one factor.

4. The method of claim 1 wherein the comparing step is performed using at least one fast fuzzy clarifier.

5. The method of claim 1 wherein the receiving step comprises receiving a signal from one or more sensors that are in direct or indirect contact with the human.

6. The method of claim 1 wherein the classifying step comprises classifying the signal according to one of an electrophysiological index, a position index, or a movement index.

7. The method of claim 1 wherein the delivering step comprises delivering a computer program instruction to a computing device via a computer interface.

8. A computer-readable carrier containing program instructions thereon that are capable of instructing a computing device to:
receive a signal indicative of physical or mental activity of a human;
use adaptive neural network based pattern recognition to identify and quantify a change in the signal;
classify the signal according to a response index to yield a classified signal;
compare the classified signal to data contained in a response database to identify a response that corresponds to the classified signal; and
deliver an instruction to implement the response.

9. The carrier of claim 8 wherein the instructions are further capable of instructing the device to process the signal to identify one or more of a cognitive state of the human, a stress level of the human, physical movement of the human body, body position changes of the human, and motion of the larynx of the human.

10. The carrier of claim 8 wherein the instructions relating to the use of adaptive neural network based pattern recognition further comprise instructions that are capable of causing the device to:
identify at least one factor corresponding to the signal; and
weight the signal in accordance to the at least one factor.

11. The carrier of claim 8 wherein the instructions relating to comparing the classified signal are further capable of instructing the device to use at least one fast fuzzy clarifier.

12. The carrier of claim 8 wherein the instructions relating to receiving a signal further comprise instructions capable of causing the device to receive a signal from one or more sensors that are in direct or indirect contact with the human.

13. The method of claim 8 wherein the instructions relating to classifying the signal further comprise instructions capable of instructing the device to classify the signal according to one of an electrophysiological index, a position index, or a movement index.

14. The method of claim 8 wherein the instructions relating to delivering further comprise instructions capable of instructing the device to deliver a computer program instruction to a computing device via a computer interface.

15. A system for causing an intended event to occur in reaction to human sensing data, comprising:
a means for receiving a signal indicative of physical or mental activity of a human;
a means for using adaptive neural network based pattern recognition to identify and quantify a change in the signal;
a means for classifying the signal according to a response index to yield a classified signal;
a means for comparing the classified signal to data contained in a response database to identify a response that corresponds to the classified signal; and
a means for delivering an instruction to implement the response.
